(19) United States
(12) Patent Application Publication
(10) Pub. No.: US 2007/0010527 A1
(43) Pub. Date: Jan. 11, 2007

(54) 4-Anilino-3-quinolinecarbonitriles for the treatment of cancer

(76) Inventor: Frank Charles Boschelli, New City, NY (US)

Correspondence Address: FITZPATRICK CELLA (WYETH), 30 ROCKEFELLER PLAZA, NEW YORK, NY, 10112-3800, US

(21) Appl. No.: 11/473,540

(22) Filed: Jun. 22, 2006

Related U.S. Application Data
(60) Provisional application No. 60/693,671, filed on Jun. 24, 2005.

(51) Int. Cl. A61K 31/496

(52) U.S. Cl. 514/253.06

(57) ABSTRACT

The present invention is directed to a method of preventing treating and/or inhibiting cancer using compounds of formula (I): 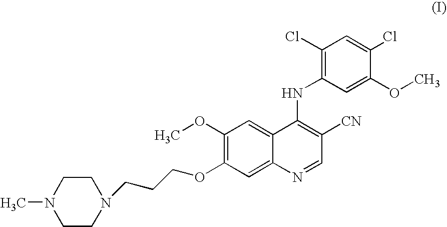 or a pharmaceutically acceptable salt thereof This invention is also directed to pharmaceutical compositions containing compounds of formula (I).

This application claims the benefit of U.S. Provisional Application No. 60/693,671, filed Jun. 24, 2005.

### BACKGROUND OF THE INVENTION

1. Field of the Invention

The present invention is directed to a method of using 4-(2,4-dichloro-5-methoxy-phenylamino)-6-methoxy-7-[3-(4-methyl-piperazin-1-yl)-propoxy]-quinoline-3-carbonitrile (SKI-606), the compound of formula (I), to treat cancer and a composition containing the same.

2. Related Background Art

Various 4-anilino-3-quinolinecarbonitriles derivatives have been shown to have anti-tumor activity that may make them useful as chemoagents in treating various cancers, including pancreatic, lymphatic and prostate cancers. U.S. Pat. Nos. 6,002,008, 6,384,051, 6,432,979 and 6,617,333 disclose certain 4-anilino-3-quinolinecarbonitriles derivatives that are shown to possess anti-tumor activity.

The protein tyrosine kinases consist of functionally related receptor and nonreceptor signaling enzymes regulating cell growth, activation, differentiation, development, and transformation through phosphorylation of specific tyrosine residues. The receptor tyrosine kinases, such as epidermal growth factor receptor (EGFR), consist of an extracellular ligand binding domain, a single transmembrane domain and an intracellular tyrosine kinase domain. The nonreceptor tyrosine kinases, such as Src and Abl, are soluble cytoplasmic enzymes with multiple regulatory and protein-binding domains.

The Src tyrosine kinase family is a group of 9 nonreceptor tyrosine kinases defined by both functional and sequence similarity. Three members of this family are widely expressed: Src, Yes, and FynB. The other 6 members, Lck, Lyn, FynT, Fgr, Hck, and Blk, are predominantly expressed in hematopoietic cells. Extensive reviews on structure and function of nonreceptor protein tyrosine kinases and their relevance in human cancers have been published.

The Src nonreceptor protein tyrosine kinase is the prototype of the Src family. Src is a key downstream component of pathways mediated by growth factor receptors and G-protein coupled receptors, and is believed to coordinate signals from these various pathways. The list of intracellular target proteins known to be phosphorylated by Src-family kinases is large and continues to grow, including integrins, adhesion kinases, cadherins, stat3, cortactin, ezrin, focal adhesion proteins (FAK), and many others.

Src is upregulated in most cancers including the vast majority of pancreatic, melanoma, head and neck, and ovarian cancers. Src is also activated in prostatic tumor lines. Src mRNA levels were higher in human parotid tumors compared to normal tissue samples. Barrett's esophagus and esophageal carcinomas also overexpress Src.

Since the c-Src tyrosine kinase is a determinant of malignant cellular behavior in a variety of human cancers, we sought to determine the effect of suppressing c-Src expression on pancreatic adenocarcinoma chemosensitivity to gemcitabine.

Based on the above observations, compounds that inhibit Src may be useful in treating patients with these and other cancers. 4-(2,4-Dichloro-5-methoxy-phenylamino)-6-methoxy-7-[3-(4-methyl-piperazin-1-yl)-propoxy]-quinoline-3-carbonitrile has been identified as useful for preventing, treating or inhibiting cancer by these criteria.

### BRIEF SUMMARY OF INVENTION

The present invention is directed to a method of preventing, treating or inhibiting cancer comprising, administering a therapeutically effective amount of a compound of formula (I): 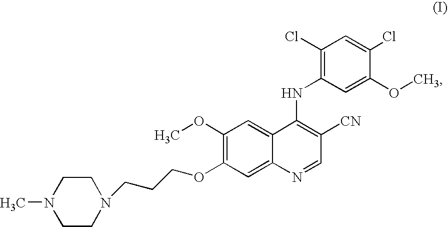
4-(2,4-dichloro-5-methoxy-phenylamino)-6-methoxy-7-[3-(4-methyl-piperazin-1-yl)-propoxy]-quinoline-3-carbonitrile, or a pharmaceutically acceptable salt thereof.

This invention is also directed to a method of treating pancreatic cancer comprising, administering a therapeutically effective amount of a compound of formula (I), or pharmaceutically acceptable salts thereof, in combination with gemcitabine, or a pharmaceutically acceptable salt thereof.

Another aspect of this invention is a pharmaceutical composition comprising an effective amount of a compound of formula (I), or a pharmaceutically acceptable salt thereof, and a pharmaceutically acceptable carrier.

### BRIEF DESCRIPTION OF THE DRAWINGS

FIG. 1. Shows the response of the head and neck line HN5 to 4-[(2,4-dichloro-5-methoxyphenyl) amino]-5-methoxy-7-[3-(4-methyl-1-piperazinyl) propoxy]-3-quinoline carbonitrile.

### DETAILED DESCRIPTION OF THE INVENTION

The term “cancer” refers to any malignant growth or tumor caused by abnormal and uncontrolled cell division. It may spread to other parts of the body through the lymphatic system or the blood stream. For the purposes of the method of treating cancer described in this application, cancer includes pancreatic, lymphatic and prostate cancers.

Pharmaceutically acceptable salts, for example, are those derived from such organic and inorganic acids as: acetic, lactic, carboxylic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

The compound of formula (I) may be provided orally, by intralesional, intraperitoneal, intramuscular or intravenous injection, infusion, liposome-mediated delivery, topical, nasal, anal, vaginal, sublingual, uretheral, transdermal, intrathecal, ocular or otic delivery. In order to obtain consistency in providing the compound of formula (I) it is preferred that the compound is in the form of the unit dose. Suitable unit dose forms include tablets, capsules and powders in sachets or vials. Such unit dose forms may contain from 0.1 to 300 mg of the compound of formula (I), and preferably from 2 to 100 mg. Still further preferred unit dosage forms contain 50 to 150 mg of the compound of formula (I). The compound of formula (I) can be administered orally. It may be administered from 1 to 6 times a day, more usually from 1 to 4 times a day. The effective amount will be known to one of skill in the art; it will also be dependent upon the form of the compound. One of skill in the art could routinely perform empirical activity tests to determine the bioactivity of the compound in bioassays and thus determine what dosage to administer.

This invention is also directed to pharmaceutical compositions containing a therapeutically effective amount of 4-(2,4-dichloro-5-methoxy-phenylamino)-6-methoxy-7-[3-(4-methyl-piperazin-1-yl)-propoxy]-quinoline-3-carbonitrile, the compound of formula (I), or pharmaceutically acceptable salts thereof, and a pharmaceutically acceptable carrier. The carrier may be, for example, a diluent, an aerosol, a topical carrier, an aqueous solution, a nonaqueous solution or a solid carrier. The carrier may be a polymer or a toothpaste. A carrier in this invention encompasses any of the standard pharmaceutically accepted carriers, such as phosphate buffered saline solution, acetate buffered saline solution, water, emulsions such as an oil/water emulsion or a triglyceride emulsion, various types of wetting agents, tablets, coated tablets and capsules. The compositions containing the compound of formula (I) may be formulated with conventional excipients, such as a filler, a disintegrating agent, a binder, a lubricant, a flavoring agent or a color additive.

When provided orally or topically, such compounds would be provided to a subject by delivery in different carriers. Typically, such carriers contain excipients such as starch, milk, sugar, certain types of clay, gelatin, stearic acid, talc, vegetable fats or oils, gums, or glycols. The specific carrier would need to be selected based upon the desired method of delivery, for example, phosphate buffered saline (PBS) could be used for intravenous or systemic delivery and vegetable fats, creams, salves, ointments or gels may be used for topical delivery.

The compound of formula (I) may be delivered together with suitable diluents, preservatives, solubilizers, emulsifiers, adjuvants and/or carriers useful in treatment or prevention of neoplasm. Such compositions are liquids or lyophilized or otherwise dried formulations and include diluents of various buffer content (for example, Tris-HCl, acetate, phosphate), pH and ionic strength, additives such as albumins or gelatin to prevent absorption to surfaces, detergents (for example, TWEEN 20, TWEEN 80, PLURONIC F68, bile acid salts), solubilizing agents (for example, glycerol, polyethylene glycerol), anti-oxidants (for example ascorbic acid, sodium metabisulfate), preservatives (for example, thimerosal, benzyl alcohol, parabens), bulking substances or tonicity modifiers (for example, lactose, mannitol), covalent attachment of polymers such as polyethylene glycol, complexation with metal ions, or incorporation of the compound into or onto particulate preparations of hydrogels or liposomes, micro-emulsions, micelles, unilamellar or multilamellar vesicles, erythrocyte ghosts, or spheroblasts. Such compositions will influence the physical state, solubility, stability, rate of in vivo release, and rate of in vivo clearance of the compound or composition. The choice of compositions will depend on the physical and chemical properties of the compound capable of treating or preventing a neoplasm.

The compound of formula (I) may be delivered locally via a capsule that allows a sustained release of the compound over a period of time. Controlled or sustained release compositions include formulation in lipophilic depots (for example, fatty acids, waxes, oils).

The present invention further provides a method of using the compound of formula (I) as an active therapeutic substance for preventing treating and/or inhibiting cancer. Based upon the results obtained and presented herein, SKI-606 is useful in preventing, treating or inhibiting cancers by suppressing proliferation of malignant cells. SKI-606 inhibits Src catalyzed phosphorylation of intercellular proteins associated with cell proliferation. Therefore, dosing a human with a therapeutically effective amount of SKI-606 can prevent or inhibit the formations of cancers by suppressing proliferation, or can treat a human already suffering from a cancer by preventing or inhibiting further growth of tumors and/or causing a reduction in size or the eradication of tumors. For the purpose of this invention the term “inhibition” refers to retardation, suppression or stopping of malignant cell proliferation, presumably by blocking, or suppressing phophorylation catalyzed by Src. For the purposes of this invention the term “preventing” refers to averting or forestalling the development of malignant or tumoric growths by prophylatic treatment, or to impede, inhibit or cease further progression of the disease. For the purposes of this invention the term “treating” refers to administering to a patient in need thereof a therapeutically effective amount of SKI-606 in order to prophylactically prevent the development of a cancer, to inhibit or cease the progression of a cancer, or a malignant or tumoric growth, to reverse the progression of a cancer, or a malignant or tumoric growth, or to eradicate a cancer, or a malignant or tumoric growth.

The present invention further provides a method of treating cancer in humans, which comprises administering to the infected individual an effective amount of 4-(2,4-dichloro-5-methoxy-phenylamino)-6-methoxy-7-[3-(4-methyl-piperazin-1-yl)-propoxy]-quinoline-3-carbonitrile, a pharmaceutically acceptable salts thereof, or a pharmaceutical composition containing the same. The dose provided to a patient will vary depending upon what is being administered, the purpose of the administration, the manner of administration, and the like. A “therapeutically effective amount” is an amount sufficient to cure or ameliorate symptoms of a cancer.

The compound of formula (I) may be delivered alone or in combination with other compounds used to treat cancer. Such compounds include but are not limited to imatinib mesylate (GLEEVEC), hydroxyurea, IFN-\acute{α}, cytotoxic agents, chemotherapeutic agents, NSAIDS, gemcitabine, EGFR inhibitors, MEK inhibitors, famesyltransferase, gemcitabine, avastin or wortmannin, or pharmaceutically acceptable salts thereof.

A preferred embodiment of the method of the present invention comprises administering a therapeutically effective amount of the compound of formula (I) in combination with a therapeutically effective amount of gemcitabine or avastin. More preferably the compound of formula (I) is administered in combination with gemcitabine.

Another preferred embodiment of the method of the present invention comprises administering a therapeutically effective amount of the compound of formula (I) in combination with a therapeutically effective amount of gemcitabine and avastin.

Another preferred embodiment of the method of the present invention involves administering the compound of formula (I), or a pharmaceutically acceptable salt thereof, in combination with gemcitabine and avastin, or pharmaceutically acceptable salts thereof, to treat pancreatic cancer.

A preferable compound for practicing the method and/or for use in a composition of this invention is 4-(2,4-dichloro-5-methoxy-phenylamino)-6-methoxy-7-[3-(4-methyl-piperazin-1-yl)-propoxy]-quinoline-3-carbonitrile and a pharmaceutically acceptable salt thereof.

The compound of formula (I) is prepared according to the methods of U.S. Pat. No. 6,002,008, and such methods are herein incorporated by reference.

Reactions are performed in a solvent appropriate to the reagents and materials employed and suitable for the transformation being effected. It is understood by those skilled in the art of organic synthesis that the various functionalities present on the molecule must be consistent with the chemical transformations proposed. When not specified, order of synthetic steps, choice of protecting groups and deprotection conditions will be readily apparent to those skilled in the art. In addition, in some instances, substituents on the starting materials may be incompatible with certain reaction conditions. Restrictions pertinent to given substituents will be apparent to one skilled in the art. Reactions were run under inert atmospheres where appropriate.

The compound of Formula (I), is readily obtained by treatment of 7-(3-chloropropoxy)-4-[(2,4-dichloro-5-methoxyphenyl)amino]-6-methoxy-3-quinolinecarbonitrile with N-methylpiperazine in the presence of sodium iodide either neat or in a solvent such as ethylene glycol dimethyl ether. The preparation of these compounds has been reported in the literature, [Boschelli, D. H., et. al., J. Med. Chem., 44, 3965 (2001)], hereby incorporated by reference.

### DETAILED DESCRIPTION OF THE DRAWINGS

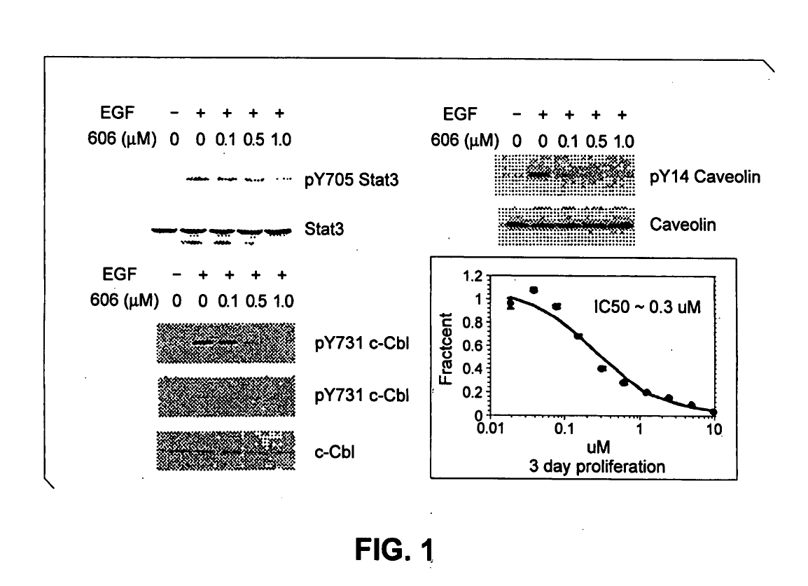

FIG. 1. Response of the head and neck tumor line HN5 to 4-[(2,4-dichloro-5-methoxyphenyl)amino]-5-methoxy-7-[3-(4-methyl-1-piperazinyl)propoxy]-3-quinolinecarbonitrile. Serum-starved HN5 cells were treated with the compound for 4 hours, after which EGF was added to 50 ng/ml for ten minutes. Lysates were analyzed for Stat3 Y705, c-Cbl Y731 and Y774 and caveolin Y14 phosphorylation. The results demonstrate that phosphorylation of caveolin Y14, c-Cbl Y731, and Y705 of Stat3 was reduced by 4-[(2,4-dichloro-5-methoxyphenyl)amino]-5-methoxy-7-[3-(4-methyl-1-piperazinyl)propoxy]-3-quinolinecarbonitrile.

Enzyme Assays

The compound of formula (I) inhibits Src catalyzed phosphorylation of a target peptide. 4-[(2,4-dichloro-5-methoxyphenyl)amino]-5-methoxy-7-[3-(4-methyl-1-piperazinyl)propoxy]-3-quinolinecarbonitrile inhibited Src catalyzed phosphorylation in a homogeneous enzyme assay (FRET/Lance format) with an IC₅₀ of 3.5 nM. A comparison of 4-[(2,4-dichloro-5-methoxyphenyl)amino]-5-methoxy-7-[3-(4-methyl-1-piperazinyl)propoxy]-3-quinolinecarbonitrile activity against various enzymes is given in Table 1.

Src and Abl Kinase Lance Assays: Recombinant human Src enzyme was obtained from PanVera (P3044). Biotinylated peptide corresponding to residues 6-20 of Cdkl was used as a substrate in the src enzyme assay (Biotin-KVEKIGEGTYGVVYK-COOH). Wild type c-Abl and v-Abl were purchased from Panvera (P3049) and Calbiochem (#102555), respectively. Biotinylated peptide for the Abl kinase assay was obtained from Synpep (Biotin-NH-KEEEAIYAAPFAKKK-COOH). For both Src and Abl kinase assays, homogeneous fluorescence resonance energy transfer kinase assays were performed using the europium/APC detection format (LANCE, Perkin Elmer). Src enzyme (10 ng) or Abl enzyme (2.5 ng c-Abl, 2.5 ng v-Abl) was mixed with biotinylated peptide (final concentration 2 μM for both substrate peptides), 50 mM Hepes (pH 7.5), 10 mM MgCl₂, 20 ug/ml BSA, and 0.001% Brij-35 (Sigma). Compound was added with a final DMSO concentration of 1%, and incubated for ten minutes at 37° C. for Src assay and 27° C. for Abl assay. The kinase reaction was initiated by addition of ATP to a final concentration of 100 μM, and incubated for 70 minutes at 37° C. for Src, and 30 minutes at 27° C. for Abl. The reaction was stopped with EDTA at a final concentration of 30 mM EDTA/25 mM Hepes (pH 7.5)/10 μg/ml BSA. The mixture was combined with Eu-labeled phosphotyrosine antibody PT66 (Perkin Elmer, AD0068) and Streptavidin Surelight-APC (Perkin Elmer, CR130-100) in 50 mM Hepes (pH 7.5)/ 20 μg/ml BSA, and incubated for 30 min according to manufacturer's specifications. The reaction was monitored on a Wallac Victor with excitation at 340 nm and emission at 665 nm. Data was analyzed with the LSW data analysis plug-in for Excel (Microsoft).

PKA, PKC, S6 kinase, CAMKII and p38 kinase assays. The Scintiplate assay is used for the kinase assay with PKA (Upstate #14-114), PKC (Upstate #14-232) and S6K (Upstate #14-333). An ELISA format is used for CAMK-II (Upstate #14-217 and p38 (Recombinant protein produced and purified in house)

Scintiplate Assay

96 well SA Cov Scintiplate (#1450-551) plates are washed 4 times (250 λ) in PBS (0.1% Triton X100) prior to the kinase assay.

60 μl mix* (See following table)
20 μl compounds (in 10 % DMSO)

20 min preincubation
20 μl substrate 1 uM (see following table)

The volumes showed in the following table are for one 96 well plate.

| Kinase | PKA | PKC | S6K |
|---|---|---|---|
| # Wells | 96.0 | 96.0 | 96.0 |
| # Reactions | 110.0 | 110.0 | 110.0 |
| Total Vol | 6600.0 | 6600.0 | 6600.0 |
| H₂O | 5452.5 | 5321.0 | 5343 |
| 10xbuffer | 1100.0 | 1100.0 | 1100.0 |
| DTT 1M | 30.0 | 30.0 | 30.8 |
| ATP 1 mM | 11.0 | 11.0 | 11.0 |
| ATP 33P | 6.0 | 6.0 | 6.0 |
| Kinases | 0.55 | 22 | 110 |
| μl/reaction | 0.005 | 0.2 | 1 |
| Lipid Activator | | 110 | |
| Substrate (1 uM) | 1463 | 1463 | 1464 |
| Reaction Time | 20′ | 30′ | 80′ |

Peptide 1323 (LSP16Bio: Bio-RTPKLARQASIELPSM: LSP-1 aa 243-258. Ser
252 is the phosphorylation site)
Peptide 1463 (PKA substrate: Bio-GRTGRRNSI)
Peptide 1464 (S6K substrate: Bio-RRRLSSLRA)
Reaction start when substrate is added.
1—Stop reaction with 20 μl of 0.5M EDTA according to the time showed in the table. Keep incubating the plate up to an hour after initiating the reaction or 80 min in the case of S6K.
2—Washes (PBS+0.1% Triton X100) six washes (250 λ/well)
3—Count (Wallac Trilux counter)
II ELISA

Following are the assay conditions for the kinases used in the ELISAs. p38 is activated with a dominant active mutant of MKK6.

| Vol (microliter) | p38 | CAMKII |
|---|---|---|
| # Wells | 96.0 | 96 |
| # Reactions | 110.0 | 110.0 |
| Total Vol. | 6600.0 | 6600.0 |
| H₂O | 5357.3 | 5464.5 |
| 10xbuffer | 1100.0 | 1100.0 |
| DTT 1M | 30.0 | 30.0 |
| 100 mM ATP | 2.8 | 3.3 |
| Kinases (Vol or | 60 nM | 2 |
| final conc) | | |
| μl/reaction | | 0.02 |
| Calmodulin(1 mg/ml) | | 44 |
| CaCl2(1 M) | | 11 |
| | 1738 | 1323 |
| Reaction time | 30′ | 30′ |

Peptide 1738 (MK2 T334: Bio-QSTKVPQTPLHTSRVL)
Peptide 1895 (Gastrin 1-17: Bio-KKEGPWLEEEEEAYGWMD)
The kinase assay is performed in the same fashion as the scintiplate assay: See step 1 through 3 above. The detection plate is a Nunc MaxiSorb precoated with Amersham goat anti-GST antibody 1:400 in PBS at 100 ul/well for 2 hours. The reaction plate is a Costar polysterene plate preblocked with blocking buffer at 0.1% Gelatin for two hours with 200 ul/well.
Prepare ERK/ADB mix as follows:
10 ul GST-ERK2 per ml of ADB
Put 60 ul of ERK/ADB mix into negative control wells (Row 12)
Prepare MEK/ERK/ADB mix (MEA) by adding active MEK1
Put 60 ul/well MEA into assay plate.
Add 20 ul cmpd 10% DMSO
Prepare 5× ATP/ADB by adding 500 uM ATP to ADB.
20 ul ATP/ADB to each well to start reaction. Incubate 1 hr at 30 C.
Stop reaction by adding 20 ul 0.5M EDTA to each well.
4—Phospho peptide detection:
Antibodies: For the detection of phosphorylated peptide, affinity purified polyclonal phospho-specific antibodies 60521 and 64273 are used against 1323 and 1738 respectively.
Add 100 μl of blocking buffer supplemented with 1% BSA with purified 60521 (0.46 mg/ml) at 1:20000 and anti rabbit-Eu (PE #ADO 105) at 1:4000 or purified 64273 at 1:4000 and anti-rabbit-Eu at 1:2000 for the detection of phosphorylated 1323 and 1738 respectively.
Add 100 μl of blocking buffer supplemented with 1% BSA with anti-phospho tyrosine PT100 (Cell Signaling #9411) at 1:1000 and anti-mouse-Eu (PE #AD0124) for the detection of phosphorylated 1895.
   Incubate 1 hour RT (Shaker)
   Wash 6×250 μl PBS 0.1% Tween 20
   Add 100 μl Enhancement Solution (Wallac Cat#1244-105)
   Incubate 10 min RT (Shaker)
   Read on Victor II (HTS Europium protocol on our reader)
   BUFFERS
   Kinase buffer 10×:
   200 mM Hepes pH 7.5, 100 mM MgCl₂
   ADB 1×:
   20 mM MOPS 7.2, 25 mM p-glycerophosphate, 5 mM EGTA, 1 mM Na orthovanadate, 20 mM MgCl₂, 1 mM DTT
   Blocking Buffer
   10 mM MOPS 7.5, 150 mM NaCl, 0.05% Tween 20, 0.1% Gelatin,0.02% NaN₃ Raf/MEK Kinase ELISA
   Reagents: Sf9 insect cell lysate containing full length 6his-tagged recombinant human c-Raf. (Specific Activity: ˜200 U/ml). Human Non-active Mek-1-GST and human GST-MAP kinase (recombinant proteins produced in E. coli).
   Rafl Kinase Cascade Assay Procedure
   Raf-1 (c-Raf) is used to phosphorylate and activate inactive GST-MEK1 which then can phosphorylate and activate inactive p42 GST-MAPK, which subsequently is measured for phosphorylation of the TEY sequence (aa's 202-204) by a phospho-specific antibody from Sigma (cat. #77439219041)
   Kinase Assay Protocol
   Stock Solutions
   Raf Assay
   1. Assay Dilution Buffer (ADB): 20 mM MOPS, pH 7.2, 25 mM β-glycerol phosphate, 5 mM EGTA, 1 mM sodium orthovanadate, 1 mM dithiothreitol.
   2. Magnesium/ATP Cocktail: 500 μM cold ATP and 75 mM magnesium chloride in ADB.
   3. Active Kinase: Human Active c-Raf: Use at 0.4U per assay point.
   4. Non-active GST-MEK 1: Use at 0.1 μg per assay point.
   5. Non-active GST-p42 MAP Kinase: Use at 1.0 μg per assay point.
   ELISA
   1. TBST-Tris (50 mM, pH 7.5), NaCl (150 mM), Tween-20 (0.05%)
   2. Superblock (Pierce)
   3. Anti-GST Ab (Pharmacia)
   4. Anti-Phospho MAPK (Sigma)
   5. Anti-Mouse Ab/Europium conjugate (Wallac)
   Assay Procedure
   First Stage: c-Raf Dependent Activation of GST-MEK and GST-MAPK
   1. Add 20 ml of ADB per assay (i.e. per well of a 96 well plate)
   2. Add 10 ml of 0.5 mM cold ATP and 75 mM magnesium chloride in ADB.
   3. Add 2 ml of c-Raf (0.4 U/assay), in conjunction with 1.6 ml non-active MEK1 (0.4 mg/assay).
   4. Add 4 ml of non-active GST-p42 MAP Kinase (1.0 mg/assay).
   5. Incubate for 60 minutes at 30° C. in a shaking incubator.
   6. Transfer this mixture to an anti-GST Ab coated 96 well plate (Nunc Immunosorb plates coated o/n with a-GST, then blocked with Pierce Superblock).
   7. Incubate for 60 minutes at 30° C. in a shaking incubator
   8. Wash 3× with TBST, add Anti-Phospho MAPK (Sigma) (1:3000)
   9. Incubate for 60 minutes at 30° C. in a shaking incubator
   10. Wash 3× with TBST, add Anti-Mouse Ab/Europium conjugate (Wallac) (1:500)
   11. Incubate for 60 minutes at 30° C. in a shaking incubator
   12. Wash 3× with TBST, Read plates in Wallac Victor model Plate Reader.
   KDR kinase assay.
   Materials
   1) Nunc MaxiSorb 96F ELISA VWR 62409-024
   2) Peptide substrate: poly(Glu4-Tyr), Sigma (P-0275): Prepare 5 mg/ml stock in water.
   3) TBS: BupH TBS Pierce (#28376); 25 mM Tris pH 7.2, 150 mM NaCl final
   4) TBST: Wash Buffer=TBS+0.05% Tween-20: For 500 ml: TBS (above)+2.5 ml of 10% Tween (made in TBS).
   5) Compound: Prepared in DMSO. Store compounds at −80C.

6) 5× KDR Kinase Buffer:

| 5X | 1X | For 50 ml of 5X |
|---|---|---|
| 20 mM HEPES pH 7.4 | 4 mM HEPES | 1 ml of 1 M HEPES |
| 5 mM MnCl2 | 1 mM MnCl2 | 1.25 ml of 200 mM MnCl2 |
| 100 uM Na3VO4 | 20 uM Na3VO4 | 0.1 ml of 50 mM Na3VO4 |

7) Enzyme diluent: 0.1% BSA in 4 mM HEPES, pH 7.4

8) 2.5X ATP [10 uM final]/MgCl2 solution in HEPES:

| 2.5X conc. | 1X in rxn | for 10 ml of 2.5X |
|---|---|---|
| 25 uM ATP | 10 uM ATP | 0.25 ml of 1 mM ATP |
| 25 mM MgCl2 | 10 mM MgCl2 | 1 ml of 250 mM MgCl2 |
| 10 mM HEPES | 4 mM HEPES | 0.1 ml of 1 M HEPES |
| | | 8.65 ml water |

9) 2.5×/MgCl2 solution in HEPES (no ATP):

| 2.5X conc. | 1X in rxn | for 5 ml of 2.5X |
|---|---|---|
| 25 mM MgCl2 | 10 mM MgCl2 | 0.5 ml of 250 mM MgCl2 |
| 10 mM HEPES | 4 mM HEPES | 0.05 ml of 1 M HEPES |
| | | 4.45 ml water |

10) ATP (FW 551): AmershamPharmacia#27-2056-01 (25 umol): 100 mM stock
11); 250 mM MgCl2: Sigma M-8266, anhydrous, FW 95.21; 1.19 g/50 ml water
12) Assay Buffer: PerkinElmer 1244-106
13) Eu-anti-PY (PT66): PerkinElmer AD0040 (50 ug, Sigma clone PT66, anti-phosphotyrosine antibody).
14) Enhancement Solution: PerkinElmer 1244-105
Methods
1. To Nunc MaxiSorb plates, add 100 ul poly(Glu4-Tyr) peptide at 25 ug/ml in TBS. Incubate 1-4 hr at RT. [Alternative: vary final poly(Glu₄-Tyr) peptide concentration from ˜1-50 ug/ml; OR use poly(Glu₆-Ala₃-Tyr) peptide]
2. Wash peptide-containing wells 3 times with 200 ul of TBS.
3. Add to each well: 29 ul of KDR Kinase Master Mix [=KDR-IC+5× KDR kinase buffer+water (mixed 1:1:0.9)] Combine 10 ul of purified KDR-IC prep, diluted (i.e., 1:5 to 1:20 depending on prep) in 0.1% BSA/4 mM HEPES+10 ul of 5× KDR kinase buffer+9 ul of water PER reaction well. Adjust water volume accordingly if using more or less compound volume. These additions can be made with a Matrix multi-pipettor.
4. Add 1 ul of compound (50× stock in DMSO depending on desired final compound concentration) to each well (TV=30 ul at this point).
5. Incubate ˜15 min at RT to allow binding of compounds to enzyme.
4. Add 20 ul of 2.5× ATP/MgCl2/HEPES solution to sample wells. For KDR, linear range of reaction is ˜1-100 nM so 10 uM is typically used. [Alternative: final ATP concentration can be varied; use of 2.5× MgCl2/HEPES with no ATP for some reactions allows determination of the feasible low end range for any enzyme prep].
5. Incubate for 40 min at room temperature. [Alternative: 30 to 60 min assay reaction; or 37C. reaction temperature, although these make minimal difference with current batches of KDR].
6. Wash the plates 3× with 200 ul of TBST.
7. Add 75 ul of Eu-PY at 1:2000 in Assay Buffer.
8. Incubate 45-60 min @ RT.
9. Wash the plates 3× with 200 ul of TBST.
10. Add 100 ul of Enhancement Solution (equilibrated to RT).
11. Read plates on VICTOR TR-fluorescent plate reader.

Raw europium (fluorescence) counts are converted to percent inhibition and/or IC50 based on untreated (no compound) control wells using Excel templates.

TABLE 1
Activity of 4-[(2,4-dichloro-5-
methoxyphenyl)amino]-5-methoxy-7-[3-(4-
methyl-1-piperazinyl)propoxy]-3-
quinolinecarbonitrile against various kinases

| | IC₅₀ (μM) |
|---|---|
| p38 | 0.95 |
| CAMKII | 6.25 |
| PKA | 5.03 |
| PKC-a | 1.47 |
| P70^{S6K} | 6.09 |
| KDR | 7.00 |
| raf/mek | 0.50 |
| Src | 0.003.5 |
| c-Abl | 0.001 |

IC₅₀ = Concentration at which there is 50% inhibition.

Cell Proliferation Data

The compounds of formula (1) is a selective inhibitor of Src kinase family kinases and Abl kinases. Cell-based assays were also used to examine the selectivity of 4-[(2,4-dichloro-5-methoxyphenyl)amino]-5-methoxy-7-[3-(4-methyl-1-piperazinyl)propoxy]-3-quinolinecarbonitrile. Rat fibroblasts overexpressing an oncogenic form of Src, where the catalytic domain of human c-Src was inserted in place of the v-Src catalytic domain to create a fusion v-Src/human c-Src protein. Similar substitutions were made with FynB, Lck, Lyn, and Hck, other Src family members. In addition, the same cell type expressing oncogenic forms of v-Abl, insulin-like growth factor-I receptor, fibroblast growth factor receptor, platelet growth factor receptor and Her2 were also constructed. Compound activity in these cells was determined using an anchorage-independent growth assay, based on the acquisition of anchorage-independence by fibroblasts expressing oncogenic proteins. Growth under these conditions is dependent on kinase activity, and inhibition of kinase activity should block cell growth in a manner that reflects inhibition of phosphorylation of cellular target proteins. Table 2 shows proliferation data obtained for 4-[(2,4-dichloro-5-methoxyphenyl)amino]-5-methoxy-7-[3-(4-methyl-1-piperazinyl)propoxy]-3-quinolinecarbonitrile under these conditions.

TABLE 2
KINASE-DEPENDENT CELL-BASED ASSAYS

| | IC₅₀(μM) |
|---|---|
| Src | 0.1 |
| Lck | 0.02 |
| Fyn | 0.4 |
| Lyn | 0.15 |
| Hck | 0.8 |
| v-Abl | 0.1 |
| Her2 | 2 |

IC₅₀ = Concentration at which there is 50% inhibition.

Head and Neck Tumor Lines

Compounds of formula (I) are potent inhibitors of tumor cell proliferation in head and neck cell lines that overexpress Src. A representative proliferation profile of 4-[(2,4-dichloro-5-methoxyphenyl)amino]-5-methoxy-7-[3-(4-methyl-1-piperazinyl)propoxy]-3-quinolinecarbonitrile with the HN5 line is shown in FIG. 1, along with evidence for inhibition of phosphorylation of downstream target proteins.

Description of Biological Test Procedures

Standard growth conditions:

For the experiments described in Table 3, 1000 cells were plated in each well of a 96 well cell culture dish on day 0 in the standard growth medium for that particular cell line. Compound was added on day 1. Relative cell number was determined on day 4.

CellTiter-Glo Method

The CellTiter-Glo luminescent cell viability assay (Promega #G7573) was used where cells were lysed with the CellTiter Glo reagent, agitated briefly and allowed to sit at room temperature for 30 minutes prior to analyzing on a Wallac plate reader equipped for luminescence readings.

MTS Assay

Some assays were monitored with the aid of the CellTiter 96 assay (Promega #G3580). With this assay, on day 4, detection reagent was added to the 96 well plate and absorbance at 490 nM was measured.

SRB Assay

The sulforhodamine B (SRB) assay was used for certain melanoma lines. In this assay, the serum concentration in the growth medium was 5% and culture medium volume was 0.2 ml. On day 4, 0.05 ml of 50% trichloroacetic acid was added to the medium and the plate was allowed to sit at room temperature for 1 hour. The medium was decanted and the plate was washed 3 times with water. The washed plates were dried, and then 0.08 ml of SRB reagent (SRB was obtained from Sigma; 0.4% SRB in 1% acetic acid) was added. After 10 minutes at room temperature, the plates were washed with 1% acetic acid until no free red color remained in solution. The bound SRB reagent was solubilized with 0.15 ml 10 mM Tris (no acid added). After agitating for 5 minutes on a shaker, the absorbance was read at 540 nM.

In Vivo Studies

All animal studies were conducted under an approved Institutional Animal Care and Use Committee protocol. Tumor cells were suspended to 50 million cells/ml and 0.2 ml of the cell suspension was injected subcutaneously into a flank of 6-7 weeks old female nude mice (Charles River, Wilmington, Mass.). Mice with tumors larger than 200 mm³ after one week were administered vehicle or compound by intraperitoneal injection at the indicated doses in 0.2 ml of vehicle containing 0.5% methylcellulose and 0.4% polysorbate 80 (Tween 80).

Table 3 summarizes the proliferation assay results obtained upon treating T-cell leukemia, prostate, pancreatic, melanoma and head and neck tumor lines with 4-(2,4-dichloro-5-methoxy-phenylamino)-6-methoxy-7-[3-(4-methyl-piperazin-1-yl)-propoxy]-quinoline-3-carbonitrile. Also shown is the ability of 4-(2,4-dichloro-5-methoxy-phenylamino)-6-methoxy-7-[3-(4-methyl-piperazin-1-yl)-propoxy]-quinoline-3-carbonitrile administration to retard the growth of A375 melanoma subcutaneous tumor xenografts in nude mice.

TABLE 3

| | IC50 (uM) | method | |
|---|---|---|---|
| T-cell leukemia | | | |
| HSB-2 | 0.014 | MTS | J |
| Molt-4 | 8.2 | MTS | |
| Prostate | | | |
| DU145 | 1.6 | Cell-glo | |
| LnCap | 3.5 | Cell-glo | |
| Pancreatic | | | |
| MiaPaca | 1.3 | Cell-glo | |
| BxPC3 | 3.9 | Cell-glo | 4 |
| Capan | 5.9 | Cell-glo | |
| Melanoma | | | |
| A375 | 1.2 | Cell-glo | |
| A375 | 0.9 | SRB | |
| RPMI-7951 | 1.2 | SRB | |
| HS-294-T | 0.6 | SRB | |
| Head and neck | | | |
| HN5 | 0.3 | Cell-glo | |

| In vivo study | Dose | T/C | |
|---|---|---|---|
| A375 melanoma | 30 mg/kg | 39%, day 12 | Bid |
| | | | ip |
| 9 days | | | |

What is claimed is:

1. A method of preventing, treating or inhibiting cancer comprising, administering a therapeutically effective amount of a compound of formula (I): 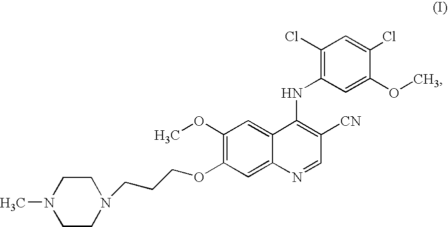
or a pharmaceutically acceptable salt thereof.

2. The method of claim 1, wherein the cancer being prevented, treated or inhibited is pancreatic cancer.

3. The method of claim 1, wherein the cancer being prevented, treated or inhibited is lymphatic cancer.

4. The method of claim 1, wherein the cancer being prevented, treated or inhibited is prostrate cancer.

5. The method of claim 1, wherein the cancer being prevented, treated or inhibited is head and neck cancer.

6. The method of claim 1, wherein the cancer being prevented, treated or inhibited is melanoma.

7. A pharmaceutical composition comprising an therapeutically effective amount of a compound of formula (I): 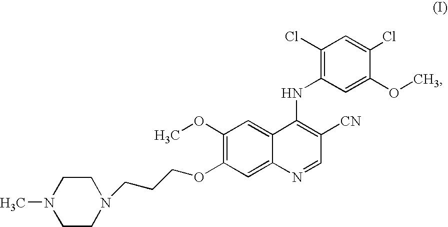
or pharmaceutically acceptable salts thereof and a pharmaceutically acceptable carrier.

8. The method of claim 1 wherein, the compound is delivered alone or in combination with one or more other compounds used to treat cancer.

9. The method of claim 8, wherein the other compounds are selected from imatinib mesylate, hydroxyurea, IFN-d, cytotoxic agents, genfitanib, gemcitabine, avastin and wortmannin, or pharmaceutically acceptable salts thereof.

10. The method of claim 9, wherein the other compound is gemcitabine.

11. The method of claim 9, wherein the other compounds are gemcitabine and avastin.

12. A method of treating pancreatic cancer comprising, administering a therapeutically effective amount of a compound of formula (I): 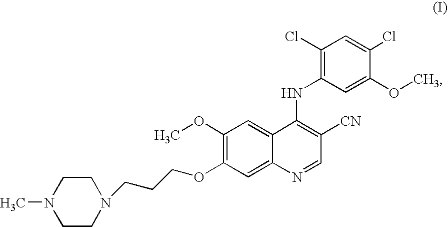
or a pharmaceutically acceptable salt thereof, in combination with gemcitabine, or a pharmaceutically acceptable salt thereof.

13. The method of claim 12, further comprising administering avastin, or a pharmaceutically acceptable salt thereof.
